(19) United States
(12) Patent Application Publication
(10) Pub. No.: US 2017/0269096 A1
(43) Pub. Date: Sep. 21, 2017

(54) REACTION SYSTEM AND USING METHOD THEREOF

(71) Applicants: NATIONAL CHENG KUNG UNIVERSITY, Tainan (TW); NATIONAL CHENG KUNG UNIVERSITY HOSPITAL, Tainan (TW)

(72) Inventors: WEI-LUN HUANG, Tainan (TW); WU-CHOU SU, Tainan (TW); HAI-WEN CHEN, Tainan (TW); YU-MIN YEH, Tainan (TW); WEI-PANG CHUNG, Tainan (TW); TE-FU YEH, Tainan (TW); HSISHENG TENG, Tainan (TW); CHIAO-YI TENG, Tainan (TW); LIANG-CHE CHEN, Tainan (TW); CHUNG-JEN CHUNG, Tainan (TW)

(21) Appl. No.: 15/462,920

(22) Filed: Mar. 20, 2017

Related U.S. Application Data
(60) Provisional application No. 62/311,194, filed on Mar. 21, 2016.

(51) Int. Cl. G01N 33/58; G01N 33/50

(52) U.S. Cl. CPC G01N 33/588; G01N 33/5011

(57) ABSTRACT

A reaction system comprises at least one additive and at least one reaction base-plane for augmenting chemical reaction, photoelectrochemical reaction, photochemical reaction or electrochemical reaction. The reaction system further comprises at least one reaction substrate carried out to the chemical reaction, the photoelectrochemical reaction, the photochemical reaction or the electrochemical reaction with the at least one additive and the at least one reaction base-plane. The at least one additive is a kind of reaction enhancer added in the reaction system to improve, augment or accumulate the effeteness of at least one reaction result.

### CROSS REFERENCE TO RELATED APPLICATION

This application claims the benefit of U.S. Provisional Application No. 62/311,194, filed on Mar. 21, 2016, the contents of which are adopted herein by reference.

### FIELD

The present disclosure relates to a reaction system and an using method thereof. More particularly, the present disclosure relates to a reaction system comprising at least one additive and at least one reaction base-plane for augmenting chemical reaction, photoelectrochemical reaction, photochemical reaction, electrochemical reaction or any combination thereof.

### BACKGROUND

Fluorescence, luminescence, spectroscopy, optical density or colors is the typical applications of optical methods in current in vitro diagnostic technique by using a light as detecting light sources or signal sources. The luminate types comprise chemiluminescence, bioluminescence or photoluminescence. Moreover, colorimetric method is the color reaction of a marker with a specific reagent. A color sensor or a photodiode can be used to obtain the color change information or the amount of light reflection to effectively improve the accuracy of the test. Spectral absorption method is a typical method to analyze substance type or substance amount. The biochemical analyzer in in vitro diagnosis is the classical uses to analyze the contents of different substances, for example serum, urine or cerebrospinal fluid. Scattering method is usually used to determine size and amount of particles, the same method can also be used to analyze the size and the structure of cells. However, the current in vitro diagnostic technique have problems of weak signal or insufficient sensitivity by using the light as the detecting sources or the signal sources. The problems cause that the test results cannot be obtained quickly and accurately, or show false positives or false negative results.

The small-molecule anticancer drugs can be classified by mechanism into antimitotic agents, alkylating agents, DNA intercalating agents, topoisomerase inhibitor, DNA cleaving agents or antimetabolites agents. Some of the drugs as well as radiation therapies can produce free radicals or peroxide compounds to attack cancer cell DNA to cause the cancer cell DNA in a wrong structure or a large number of DNA broken so that the cancer cell DNA cannot be repaired, synthesis and replication to treating cancer. However, the present anticancer drugs are expensive, burden to human body, and hard to track utility and distribution of the drug in vivo after the drug administration. Therefore, a new drug development is urgent need.

Wastewater treatment mostly treats domestic sewage and industrial wastewater by physical, biological or chemical ways to separate solid contaminants from water, and reduce organic contaminants and nutrients in water, thereby reducing environmental pollution. Wastewater treatment usually comprises primary treatment, secondary treatment or tertiary treatment. The primary treatment is removing solid waste, oil, sand, hard particles and other precipitatable material in the sewage. The whole process is mechanical. The secondary treatment is decomposing organic compounds to inorganic substances. The tertiary treatment is further using to sand filter, activated carbon or microorganism to remove toxins or heavy metals. The decomposing waste, especially in wastewater treatment has always been a major problem. General wastewater, industrial wastewater, and even to oil pollution are required to be effective and environmental protection technology.

The conventional metallic semiconductor quantum dots, for example titanium dioxide (TiO₂) or cadmium sulfide (CdS), can be used for converting light energy to generate electron-hole pairs or redox pairs. The conventional metallic semiconductor quantum dots also can catalyze the reactant comprising, for example water, organic pollution or ammonia, to perform the oxidation-reduction reaction, and to further produce hydrogen or achieve decontamination to solve energy or environmental problems by the different reactants. Furthermore, the free radicals or peroxides comprising O₂^{}, OH^{} or H₂O₂ are produced in a photochemical reaction process, which can inhibit tumor growth or reproduction of bacteria. The electron-hole pairs generated by a light supplement can be recombined to release photons and to carry out a photoluminescence reaction used in detection of the target for assisting diagnosis.

The conventional metallic semiconductor quantum dots have several disadvantages. (1) In the application of solar energy, the absorption range of sunlight is in ultraviolet wavelength from 190 nanometer to 380 nanometer. The absorbed energy is only 4 percentage of sunlight energy, and unable to effectively improve the efficiency. (2) Since the TiO₂ quantum dots are very stable, and not easy to change the electronic properties by surface modification; therefore, the TiO₂ quantum dots are not conducive to biomedical diagnosis, treatment of specific target design, and other applications in development related components. (3) In the treatment, since the penetration of the ultraviolet light to skin is imperfect, the ultraviolet light excites a low concentration of free radicals in the human body by carrying out the photochemical reactions. Therefore, the inhibition effect of tumor growth or bacterial growth is limited. (4) In the diagnosis, the TiO₂ quantum dots are a very stable, the electron-hole pairs generated under the light irradiation can be stably stored in the TiO₂ quantum dots, it is difficult to re-combine to release photons, and therefore the target position, for example a specific cell, tissue or microbial, cannot be effectively detected for assisting the diagnosis.

Moreover, another general metallic semiconductor quantum dots, the CdS quantum dots, have several disadvantages. (1) Although the light absorption range can be extended to a wavelength range from 190 nanometer to 900 nanometer, the Cds quantum dots are easily oxidized by the generated electron-hole pairs under a light irradiation, and occurred photo corrosion. The catalysis reaction with the CdS quantum dots cannot be performed stably. (2) In the treatment, the red to near-infrared light can be used as an optimized light source, however, the generation efficiency of the free radicals or peroxides of the CdS quantum dots is too low to treatment uses. (3) Metal cadmium shows high bio-toxicity, and is not suitable for diagnosis and treatment in vivo. (4) When applying to the diagnosis, since the CdS quantum dots show poor hydrophilicity, the CdS quantum dots need to perform a tedious surface modification before uniformly dispersing in water. The process complexity of the tedious surface modification is increased, the yield is declined, the costs are increased, and the stability in water phase is affected, all of the disadvantages are against the biomedical applications of the CdS quantum dots. (5) The CdS quantum dots is difficult to be connected with biological molecules, for example antibodies, proteins, nucleic acids or lipids, resulting in a hard modification of specificity. The surface of the CdS quantum dots contains many defects which cause energy lose, and it is hard to carry out the photoluminescence under a light irradiation.

The conventional metallic semiconductor quantum dots have the congenital obstacles of material properties, comprising absorbance capacity, energy conversion efficiency, toxicity or chemical modification, all of which are insurmountable, therefore, their application scopes are limited. Thus, it is necessary to provide a reaction system comprising an enhancer and a reaction base-plane, and a method for carrying out chemical reaction, photoelectrochemical reaction, photochemical reaction or electrochemical reaction by using the same to solve the problems existing in the conventional technology as described above.

### BRIEF DESCRIPTION OF THE DRAWINGS

Implementations of the present technology will now be described, by way of example only, with reference to the attached figures. The aspect of the present disclosure can be better understood with reference to the following figures. The components in the figures are not necessarily drawn to scale with the emphasis instead being placed upon clearly illustrating the principles of the present disclosure.

FIG. 1 illustrates optical density changes of chemical reactions of trypan blue (TB) without or with graphene oxide quantum dots (GOQD), without or with an enhancer (triethanolamine, TEOA), or with a combination of the GOQD and the TEOA, under a light supplement.

FIG. 2 illustrates fluorescent intensity changes of reaction systems of Amplex® Red reagent dissolving in Milli-Q ultrapure water (MQ) without or with graphene oxide quantum dots (GOQD), without or with an enhancer (SF RPMI), or with a combination of the GOQD and the SF RPMI, under a light supplement.

FIG. 3 illustrates chemiluminescent intensity changes of reaction systems of a Luciferin Precursor dissolving in Milli-Q ultrapure water (MQ) without or with graphene oxide quantum dots (GOQD), without or with an enhancer (SF RPMI) or with a combination of the GOQD and the SF RPMI, under a light supplement.

FIG. 4 illustrates concentration (conc., μM) changes of a specific mediator hydrogen peroxide (H₂O₂) in reaction systems presented in FIG. 2.

FIG. 5 illustrates a linear statistics result of reaction systems of presented in FIG. 2, which is correlated well with the concentrations of graphene oxide quantum dots (GOQD) in a reaction system.

FIG. 6 illustrates a specific mediator hydrogen peroxide (H₂O₂) concentration (μM) changes in ultrapure water with an enhancer (triethanolamine, TEOA) and different doses of graphene oxide quantum dots (GOQD), under a light supplement for 10 minutes (L 10 min).

FIG. 7 illustrates a specific mediator hydrogen peroxide (H₂O₂) concentration (μM) changes in ultrapure water with different doses of an enhancer (Urea) and graphene oxide quantum dots (GOQD), under a light supplement.

FIG. 8 illustrates a specific mediator hydrogen peroxide (H₂O₂) concentration (μM) changes in ultrapure water with an enhancer (ascorbic acid, AA) and different doses of graphene oxide quantum dots (GOQD), under a light supplement for 10 minutes (L 10 min).

FIG. 9 illustrates a specific mediator hydrogen peroxide (H₂O₂) concentration (μM) changes in ultrapure water with different doses of an enhancer (co-enzyme Q10, Q10) and graphene oxide quantum dots (GOQD), under a light supplement for 10 minutes (L 10 min).

FIG. 10 illustrates a specific mediator hydrogen peroxide (H₂O₂) concentration (μM) changes in ultrapure water with different doses of an enhancer (astaxanthin, A) and graphene oxide quantum dots (GOQD), under a light supplement for 10 minutes (L 10 min).

FIG. 11 illustrates a specific mediator hydrogen peroxide (H₂O₂) concentration (μM) changes in ultrapure water without or with graphene oxide quantum dots (GOQD), without or with an enhancer (phosphate-buffered saline, PBS), or with a combination of the GOQD and the PBS, and without a light supplement (NL) or with a light supplement for 10 minutes (L 10 min).

FIG. 12 illustrates a specific mediator hydrogen peroxide (H₂O₂) concentration (μM) changes in Milli-Q ultrapure water (MQ) without or with graphene oxide quantum dots (GOQD), without or with an enhancer (KMnO₄), or with a combination of the GOQD and the KMnO₄, under a light supplement.

FIG. 13 illustrates cell viability changes (%) of lung cancer cells by treating without or with graphene oxide quantum dots (GOQD), without or with an enhancer (triethanolamine, TEOA), or with a combination of the GOQD and the TEOA, and without a light supplement (NL) or with a light supplement for 10 minutes (L 10 min).

FIG. 14 illustrates cell viability changes (%) of lung cancer cells by treating without or with graphene oxide quantum dots (GOQD), without or with an enhancer (ascorbic acid, AA), or with a combination of the GOQD and the AA, and without a light supplement (NL) or with a light supplement for 10 minutes (L 10 min).

FIG. 15 illustrates a specific mediator hydrogen peroxide (H₂O₂) concentration (μM) changes in ultrapure water with an enhancer (ascorbic acid, AA) and different doses of graphene oxide quantum dots (GOQD), and without a light supplement.

FIG. 16 illustrates a specific mediator hydrogen peroxide (H₂O₂) concentration (μM) changes in Milli-Q ultrapure water (MQ) without or with graphene oxide quantum dots (GOQD), without or with an enhancer (ascorbic acid: AA), or with a combination of the GOQD and the AA, and without a light supplement (NL) or with a light supplement for 10 minutes (L 10 min).

FIG. 17 illustrates cell viability changes (%) of lung cancer cells by treating without or with graphene oxide quantum dots (GOQD), without or with a higher dose enhancer (ascorbic acid: AA), or with a combination of the GOQD and the AA, and without a light supplement (NL) or with a light supplement for 10 minutes (L 10 min).

FIG. 18 illustrates cell viability changes (%) of colon cancer cells by treating without or with graphene oxide quantum dots (GOQD), without or with an enhancer (ascorbic acid: AA), or with a combination of the GOQD and the AA, and without a light supplement (NL) or with a light supplement for 10 minutes (L 10 min).

FIG. 19 illustrates current intensity change in ultrapure water with graphene oxide quantum dots (GOQD), with an enhancer (triethanolamine, TEOA), under an electrical energy and a light supplement.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

Several definitions that apply throughout this disclosure will now be presented.

The term “coupled” is defined as connected, whether directly or indirectly through intervening components, and is not necessarily limited to physical connections. The connection can be such that the objects are permanently connected or releasably connected. The term “outside” refers to a region that is beyond the outermost confines of a physical object. The term “inside” refers to a region that is within the outermost confines of a physical object. The term “surface” in the present disclosure refers to the outer part or uppermost layer of a material layer constituting. The term “comprising,” when utilized, means “including, but not necessarily limited to”; it specifically indicates open-ended inclusion or membership in the so-described combination, group, series and the like. The term “providing” refers to supply something needed or desired to. The term “applying” refers to put to or adapt for a special use, or to put into an action.

The term “variation” in the present disclosure means a change or a difference in condition, amount or level, or an act, a process or a result that differs from a standard or convention. The term “photoelectrochemical” is relating to, or designating an electrochemical cell or reaction in which the electrode potential or the current depends on the nature of the non-metallic semiconductor quantum dots. The variation of current intensity depends on the degree of illumination. The term “photochemical” is the branch of chemistry concerned with the chemical effects of light. Generally, this term is used to describe a chemical reaction caused by absorption of ionizing radiation, ultraviolet, visible light or infrared radiation. The term “electrochemical” is the branch of physical chemistry that studies the relationship between electricity and identifiable chemical change, with either electricity considered an outcome of a particular chemical change or vice versa. These reactions involve electric charges moving between electrodes and an electrolyte. Thus electrochemistry deals with the interaction between electrical energy and chemical change. The term “photoluminescence” is light emission from any form of matter after the absorption of infrared radiation, visible light, or ultraviolet radiation. The term “photovoltaic” refers to produce a voltage when exposed to radiant energy, especially light. The term “photocatalysis” means the alteration of the rate of a chemical reaction by light or other electromagnetic radiation. The term “additive” in the present disclosure means a substance added to a reaction system else to improve, strengthen or otherwise alter it. The term “enhancer” in the present disclosure means a substance added in a reaction system to improve, augment or accumulate the effeteness of the reaction result. The term “substrate” in the present disclosure means a material or a substance on which a reaction system acts. The term “bio-compound” is a biological material or a biological substance made up of one or more parts or elements. The term “element” in the present disclosure means a substance composed of atoms having an identical number of protons in each nucleus. Such elements cannot be reduced to simpler substances by normal chemical means. The term “orbital”, also called atomic orbital, is a mathematical function that describes the wave-like behavior of either one electron or a pair of electrons in an atom. This function can be used to calculate the probability of finding any electron of an atom in any specific region around the atom's nucleus. The atomic orbital may also refer to the physical region or space where the electron can be calculated to be present. The term “dope” or “doping” in the present disclosure means an additive used to improve the properties of a semiconductor. The doping is the intentional introduction of impurities into an intrinsic semiconductor for the purpose of modulating its electrical properties. The term “functional group” in the present disclosure means an atom or group of atoms. In particularly, the functional groups are specific groups or moieties of atoms or bonds within molecules that are responsible for the characteristic chemical reactions of those molecules. The term “administer” or “administration” in the present disclosure refers to give or provide something, for example a light supplement, to a subject or a reaction.

Graphene is a flat monolayer comprising sp²-bonded carbon atoms tightly packed into a two-dimensional honeycomb lattice. Since graphene was first isolated from graphite using sticky tape in 2004, it has attracted tremendous interest from the research community. Graphene exhibits many extraordinary physical properties, such as a high optical transparency, a large theoretical specific surface area, a high charge carrier mobility at room temperature, and an ultra-high electron conductivity. These characteristics make graphene a promising candidate for a wide range of applications in field-effect transistors, conductive electrodes for solar cells, supercapacitors, and lithium-ion batteries. These characteristics can be attributed to the fact that graphene is a zero-gap semiconductor and an atomically thin monolayer.

Graphene oxide (GO), a polymer-like graphitic semiconductor made of only carbon, oxygen, and hydrogen, has a large exposed area and can extensively be dispersed in water on the molecular scale. The GO is the intermediate state between graphene and graphite. Unlike graphite, however, the GO easily exfoliates and disperses in aqueous solution like wrinkled paper because its functional oxygen groups are hydrophilic. The structure and electronic properties of the GO change because of the composition of the oxygen bonding on graphene. Because oxygen atoms have a larger electro-negativity than carbon atoms, the GO becomes a p-doped material where the charge flow create negative oxygen atoms and a positively charged carbon grid. The GO bandgap increases with the oxidation level. Fully oxidized GO is an insulator, in contrast to partially oxidized GO and graphene that are a semiconductor and a conductor, respectively. Therefore, the oxidation level of GO can be used to tune the electronic properties depending on the application. The GO has been reported to have some unique optical properties such as photoluminescence (PL)/fluorescence (FL) and electrochemiluminescence (ECL). Furthermore, the GO is the most important precursor to prepare graphene, which has been widely applied in broad fields due to the low mass density, excellent electrical conductivity and high specific surface area.

The present disclosure is described in relation to a reaction system comprising at least one additive and at least one reaction base-plane, and an using method thereof to produce at least one chemical reaction result, at least one photoelectrochemical reaction result, at least one photochemical reaction result, at least one electrochemical reaction result or any combination thereof. The reaction system further comprises at least one reaction substrate carried out to the chemical reaction, the photoelectrochemical reaction, the photochemical reaction or the electrochemical reaction with the at least one additive and the at least one reaction base-plane.

The at least one additive is a kind of reaction enhancer. The enhancer is incorporated on the basis of its redox potentials that is encompassed by conduction and valence bands of non-metallic semiconductor quantum dots. The enhancer comprises nutrients, vitamins, alkali salts or alkali buffers, organic compounds, inorganic compounds, transition metal ions having an empty d, f, or g orbital or any combination thereof. The nutrients may comprise serum free RPMI, serum free DMEM, serum free MEMα, serum free F12, serum free L15, serum free Hybri-Care or fetal bovine serum. The vitamins may comprise ascorbic acid, co-enzyme Q10, glutathione or astaxanthin. The alkali salts or the alkali buffers may comprise phosphate-buffered saline or polysulfide. The organic compounds (which are preferably heterocyclic compounds, macrocycles, or organic compounds with hydroxyl group, carbonyl group, or nitrogen) may comprise porphyrin, chlorophyll, histamine, methanol, ethanol, triethanolamine or lactic acid. The inorganic compounds may comprise silver nitrate or sodium iodate. The transition metal ions having an empty d, f, or g orbital may comprise ferric ion, ferrous or potassium permanganate, cobalt ion, nickel ion, or any combination thereof. The enhancer can improve, augment or accumulate the variation of chemical reaction, the variation of photoelectrochemical reaction, the variation of photochemical reaction or variation of electrochemical reaction. The enhancer may be added to the reaction system in a concentration range from about 1×10⁻¹² volume per volume percentage (% v/v) to about 50% v/v or a concentration range from about 1×10⁻¹⁵ molarity (M) to about 10 M.

The serum free RPMI, the serum free DMEM, the serum free MEMα, the serum free F12, the serum free L15 or the serum free Hybri-C is a kind of serum free media. The serum free media is the usual media without fetal calf serum literally. The serum free RPMI is RPMI media without fetal calf serum, which is developed by Moore et. al. at Roswell Park Memorial Institute, hence the acronym RPMI. The RPMI media formulation is based on utilizing a bicarbonate buffering system and alterations in the amounts of amino acids and vitamins. The serum free DMEM is Dulbecco's Modified Eagle's medium (DMEM) without fetal calf serum. The DMEM is a modification of Basal Medium Eagle (BME) that contains a four-fold higher concentration of amino acids and vitamins, as well as additional supplementary components. The serum free MEMα is Minimum Essential Medium alpha (MEMα) modification media without fetal calf serum. The MEMα is one of the most widely used of all synthetic cell culture media. The serum free F12 is nutrient mixture Ham's F-12 medium (F-12) without fetal calf serum. The F-12 has higher levels of amino acids, vitamins and trace elements. Putrescine and linoleic acid are added to the F-12. The F-12 is originally designed for the serum-free growth of Chinese hamster ovary (CHO) and lung cells. The serum free L15 is Leibovitz L-15 medium (L15) without fetal calf serum. The L15 is buffered with a complement of salts, free base amino acids and galactose, so the L15 can be used under conditions of free gaseous exchange with the atmosphere. The serum free Hybri-Care is Hybri-Care Medium (ATCC® 46-X™) (Hybri-Care) without fetal calf serum. The Hybri-Care is a special medium formulated to support the growth of hybridomas and fastidious cell lines. The buffering system of 4-(2-hydroxyethyl)-1-piperazineethanesulfonic acid (HEPES) and added sodium bicarbonate (NaHCO₃) enables the Hybri-Care to be used at all stages of the hybridoma production from fusion to cloning, including single-cell subcloning. The fetal bovine serum (FBS) is the blood fraction remaining after the natural coagulation of blood, followed by centrifugation to remove any remaining red blood cells. The FBS comes from the blood drawn from a bovine fetus via a closed system of collection at the slaughterhouse. The sugar, also known as glucose, is a chemical with formula C₆H₁₂O₆.

The ascorbic acid, also known as vitamin C, is a vitamin found in food and used as a dietary supplement. The co-enzyme Q10, also known as ubiquinone or ubidecarenone, and abbreviated at times to CoQ10, CoQ or Q10, is a coenzyme that is ubiquitous in the bodies of most animals. The co-enzyme Q10 is a 1,4-benzoquinone, where Q refers to the quinone chemical group and 10 refers to the number of isoprenyl chemical subunits. The glutathione is an important antioxidant in plants, animals, fungi, some bacteria or archaea. The glutathione is capable of preventing damage to important cellular components caused by reactive oxygen species such as free radicals, peroxides, lipid peroxides or heavy metals. The astaxanthin is a keto-carotenoid. The astaxanthin is found in microalgae, yeast, salmon, trout, krill, shrimp, crayfish, crustaceans, and the feathers of some birds. The astaxanthin provides the red color of salmon meat and the red color of cooked shellfish. The astaxanthin is an antioxidant with a slightly lower antioxidant activity in some model systems than other carotenoids.

The phosphate-buffered saline (PBS) is a buffer solution commonly used in biological research. The PBS is a water-based salt solution containing disodium hydrogen phosphate, sodium chloride, or potassium chloride and potassium dihydrogen phosphate in some formulations. The osmolarity and ion concentrations of the PBS match those of the human body (isotonic). The polysulfide, a mixture of sodium sulfide (Na₂S) and sodium sulfide (Na₂SO₃), exhibits strong reducing ability for hole scavenger.

The methanol, also known as methyl alcohol, is a chemical with the formula CH₃OH (MeOH). The ethanol, also known as alcohol, is a chemical with the formula C₂H₅OH (EtOH). The lactic acid is a chemical with formula C₃H₆O₃. The triethanolamine, often abbreviated as TEA or TEOA, is a viscous organic base that is both a tertiary amine and a triol.

The silver nitrate is an inorganic compound with chemical formula AgNO₃. The silver nitrate is a versatile precursor to many other silver compounds. The sodium iodate is the sodium salt of iodic acid. The sodium iodate is an oxidizing agent, and as such it can cause fires upon contact with combustible materials or reducing agents.

The ferric ion is iron with an oxidation number of +3, also denoted iron (III) or Fe³⁺, and is usually the most stable form of iron in air. Ferrous (Fe²⁺), in chemistry, indicates a divalent iron compound (+2 oxidation state) that can be regarded as a reducing reagent. The potassium permanganate, the chemical formula KMnO₄, is a salt consisting of K⁺ and MnO⁻⁴ ions. It is a strong oxidizing agent.

The at least one reaction base-plane comprises at least one non-metallic semiconductor quantum dot. The at least one reaction base-plane is added to the reaction system in a concentration range from about 1×10⁻¹⁵ milligram per milliliter (mg/mL) to 500 mg/mL. The at least one non-metallic semiconductor quantum dot in the present disclosure has a particle size range from about 0.34 nanometer (nm) to about 100 nm, for example 0.34 nm, 0.5 nm, 1 nm, 3 nm, 5 nm, 10 nm, 15 nm, 20 nm, 25 nm, 30 nm, 35 nm, 40 nm, 45 nm, 50 nm, 55 nm or 60 nm. The at least one non-metallic semiconductor quantum dot may be constructed by a group IVA element, comprising a carbon-based material or a silicon-based material. Preferably, the carbon-based material is graphene or graphene oxide. The at least one non-metallic semiconductor quantum dot comprises graphene quantum dot or graphene oxide quantum dot. Additionally, the shape of the at least one non-metallic semiconductor quantum dot generally presents ball-shape, pillar-shape or disc-shape. The graphene oxide quantum dot preferably presents a disc-shaped structure having a thickness range from about 0.34 nm to about 20 nm, for example 0.34, 0.5, 1, 3, 5, 10, 15 or 20 nm.

Graphene quantum dots (GQDs) represent single-layer to tens of layers of graphene. Due to the GQDs exceptional properties such as low toxicity, stable photoluminescence, chemical stability and pronounced quantum confinement effect, the GQDs are considered as a novel material for biological, opto-electronics, energy or environmental applications. The GQD is becoming an advanced multifunctional material for its unique optical, electronic, spin, and photoelectric properties induced by the quantum confinement effect and edge effect. The GQDs have various important applications in bio-imaging, cancer therapeutics, temperature sensing, drug delivery, LEDs lighter converters, photo-detectors, solar cells, fluorescent material or biosensors fabrication.

The graphene oxide quantum dots (GOQDs) comprise a sp² domain that renders high carrier transport mobility as well as disordered sp³ hybridized carbon atoms, which bind to oxygen-containing functional groups at the edge sites and on the basal plane. Thus, the GOQD not only have the previous mentioned characteristics and functions of the GQDs but could also provide other benefit.

The most GQDs emit blue light under UV excitation, which seriously limits their scope of applicability. The ability to produce the GOQDs capable of emitting other colors will be highly desirable, particularly for the development of devices requiring white light emission.

Numerous methods have been developed for synthesizing the at least one non-metallic semiconductor quantum dot, comprising top-down methods or bottom-up methods. The top-down methods comprise microwave method, chemical oxidation method, reflux method, hydrothermal method, solvothermal method, electrochemical method, ultrasonic method or RF-plasma method. The bottom-up methods comprise microwave method, hydrothermal method, pyrolysis method, stepwise method, chemical vapor deposition method or self-assembly method.

The at least one non-metallic semiconductor quantum dot may comprise at least one dopant. The doping methods comprise electrochemical method, arc discharge method, hydrothermal method, calcination modification method, Hummer's method, modified Hummer's method or ammonia catalyzed dehydration method. The at least one dopant may comprise at least one IIA group element, at least one IIIA group element, at least one IVA group element, at least one VA group element, at least one VIA group element, at least one transition element having an empty d orbital or any combination thereof. The at least one dopant may preferably comprise Mg, O, N, P, B, Fe, Co or Ni element. The at least one dopant may be doped in the at least one non-metallic semiconductor quantum in a doping ratio from about 0 mole percentage (mol %) to about 50 mol %.

The at least one non-metallic semiconductor quantum further comprises the at least one functional group. The at least one non-metallic semiconductor quantum may be functionalized with the at least one functional group in a ratio from about 0 mol % to about 50 mol %. The at least one functional group may comprise at least one hydrogen atom, at least one IIIA-element functional group, at least one IVA-element functional group, at least one VA-element functional group, at least one VIA-element functional group, at least one VIIA-element functional group or any combination thereof. The at least one functional group may preferably comprise an amino group (NH₂—), a phosphite group (—PO₃), a carbonyl group (—CO), a carboxyl group (—COOH), a acyl group, a boron atom (B—), a hydrogen atom (H—), a hydroxyl group (—OH), a nitrogen atom (N—), an oxygen atom (O—), a sulfur atom (S—), a phosphorus atom (P—) or any combination thereof.

The at least one reaction substrate may comprise at least one inorganic compound, at least one organic compound, at least one bio-compound, at least one cell, at least one microbial, at least one parasite or any combination thereof. The at least one bio-compound may comprise nucleotides, DNA, RNA, lipids, amino acids, peptides, saccharides or any combination thereof. The at least cell may further comprise at least one cell derived component, comprising organelle, extracellular vesicle or inclusion body.

At least one predetermined energy may be provided to the reaction system to induce, produce, enhance or augment the reaction. The at least one predetermined energy comprises a radiation energy, a heat energy, an electrical energy, a magnetic energy or a mechanical energy. The radiation energy is administered in a wavelength range from 1 pm to 1600 nm, or any combination thereof; the radiation energy is preferably administered in a wavelength range from 1 pm to 1 nm (ionizing radiation); 10 nm-400 nm (Ultraviolet light); and especially 400 nm to 1000 nm (visible light and infrared ray), or any combination thereof. The radiation energy is administered in a power range from about 1×10⁻⁶ μW/cm² to about 100 W/cm²; the radiation energy is preferably administered in a power range from about 10 microWatt per square centimeter (μW/cm²) to about 5 Watt per square centimeter (W/cm²). The heat energy is administered in a power range from about 1×10⁻⁶ μW/cm² to about 100 W/cm²; the heat energy is preferably administered in a power range from about 10 μW/cm² to about 5 W/cm². The electrical energy is administered in an electric potential range from about 0.0001 voltage to about 500 voltage; the electrical energy is preferably administered in an electric potential range from about 0.1 voltage to about 5 voltage. Additionally, the electrical energy is administered in an current response ranging from about 1×10⁻¹⁵ Ampere per square centimeter (A/cm²) to about 100 A/cm²; the current response ranging is preferably from about 1×10⁻¹² A/cm² to about 10 A/cm². The magnetic energy is administered in a power range from about 1×10⁻⁶ μW/cm² to about 100 W/cm²; the magnetic energy is preferably administered in a power range from about 10 μW/cm² to 5 W/cm². The mechanical energy may be preferable ultrasonic energy. The mechanical energy is administered in a power range from about 1×10⁻⁶ μW/cm² to about 100 W/cm²; the mechanical energy is preferably administered in a power range from about 10 μW/cm² to about 5 W/cm².

The reaction system is used to augment the chemical reaction, the photoelectrochemical reaction, the photochemical reaction, the electrochemical reaction or any combination thereof. The photoelectrochemical reaction further comprises variation of photoluminescence reaction or variation of photovoltaic reaction. The photochemical reaction further comprises variation of photocatalysis reaction.

The chemical reaction, the photoelectrochemical reaction, the photochemical reaction, electrochemical reaction or any combination thereof may induce redox reaction, electrochromic, electrochemiluminescence, photochemiluminescence, or photoelectrochemiluminescence.

The chemical reaction, the photoelectrochemical reaction, the photochemical reaction, the electrochemical reaction or any combination thereof can be carried out with or without the enhancer which form a composition with the non-metallic semiconductor quantum dot to augment at least one reaction result, comprising color change, luminescence intensity variation, fluorescence intensity variation, optical density variation, luminescence wavelength profile variation, fluorescence wavelength profile variation, optical wavelength profile variation, current intensity change, electron response, substrate amounts decreasing, substrate activities change, substrate compounds modification or substrate functions change.

The color change, the luminescence intensity variation, the fluorescence intensity variation, the optical density variation, the luminescence wavelength profile variation, the fluorescence wavelength profile variation, the optical wavelength profile variation, the current intensity change, the electron response, substrate amounts decreasing, substrate activities change, substrate compounds modification or substrate functions change in the reaction system can be used to indicate, semi-quantify or quantify the non-metallic semiconductor quantum dot, the reaction substrates (direct or indirect) as target molecules interacted with the non-metallic semiconductor quantum dot, or the target molecules conjugated with the non-metallic semiconductor quantum dot by interactions including but not limited to covalent bond, ester bond, peptide bond, thioester bond. These can also be used for quantification, semi-quantification, or indication of the present of the target molecules which can interact with the molecules that are conjugated with the non-metallic semiconductor quantum dot for detecting the target molecules. These can also be used for quantification, semi-quantification, or indication of the present of the “reactive”, “non-reactive” or “the ratio of reactive/non-reactive” of non-metallic semiconductor quantum dot, and consequently be used for quantification, semi-quantification, or indication of the present of targets molecules which can react directly with the non-metallic semiconductor quantum dot or indirectly via specific mediators for detecting the target molecules.

The color change, the luminescence intensity variation, the fluorescence intensity variation, the optical density variation, the luminescence wavelength profile variation, the fluorescence wavelength profile variation, the optical wavelength profile variation, the current intensity change, the electron response, substrate amounts decreasing, substrate activities change, substrate compounds modification or substrate functions change in the reaction system can be occurred by the addition of the enhancers, the changes of the non-metallic semiconductor quantum dot, the different reaction substrate, the administration of the predetermined energy or the other components in the reaction system.

The enhancer can be used to enhance similar reactions to the chemical reaction, the photoelectrochemical reaction, the photochemical reaction or the electrochemical reaction that are carried out by photochemical reporter agents or electrochemical reporter agents other than the non-metallic semiconductor quantum dot. The photochemical reporter agents or the electrochemical reporter agents further comprises metallic semiconductor quantum dots, non-semiconductor quantum dots, dye molecules, sensitizers, energy absorbing substances, energy directing molecules, energy gathering molecules or any combination thereof. The metallic semiconductor quantum dots comprise CdSe quantum dots, CdTe quantum dots, CuS quantum dots, CuSe quantum dots, PbS quantum dots, ZnO quantum dots, SnO₂ quantum dot and for ternary compound such as CuInS₂ quantum dots, CuInSe₂ quantum dots, InAsN quantum dots, TiO₂ quantum dots, GaP quantum dots, or GaAs quantum dots. The non-semiconductor quantum dots comprise TiO₂, PbO, Cu₂O, CuO, WO₃, SnO₂, Bi₂O₃, Fe₂O₃, BiVO₄, SrTiO₃, BaTiO₃, FeTiO₃, KTaO₃, InAsN, GaP, GaAs, or MnTiO₃.

The detecting can be carried out in forms of lateral flow, micro-chip assay, ELISA, well-base assay, cuvette-based assay, semiconductor-based sensor chip, photomultiplier, charge-coupled device based assay, complementary metal-oxide semiconductor based assay, potentiostat/galvanostatt/cyclic voltammetry instruments, electrochemical analyzers, electrochromic analyzers, electrochemiluminescence analyzers, electrochemiluminescence analyzers, photochemiluminescence or photoelectrochemiluminescence analyzsers, but it is not limited thereto.

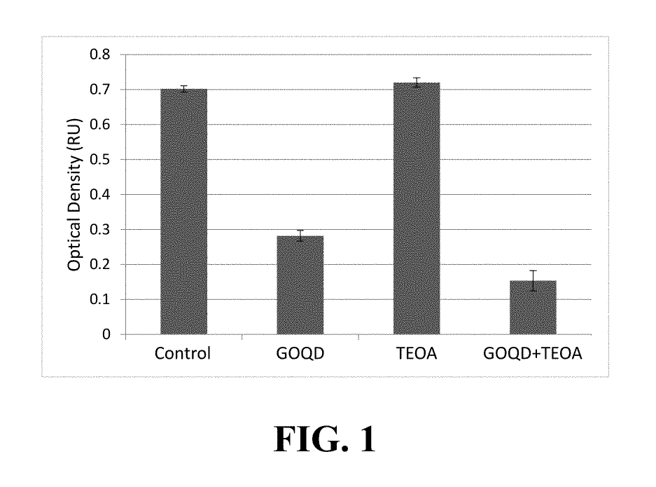

FIG. 1 illustrates optical density changes of chemical reactions of trypan blue (TB) without or with the GOQD at the concentration of 0.08 mg/mL, without or with an enhancer (triethanolamine, TEOA) at the concentration of 0.5% v/v, or with a combination of the GOQD and the TEOA, under a light supplement (55 mWatt/cm², 30 min). The chemical reaction with trypan blue (TB) is carried out in the presence of the GOQD or with the enhancer under a light supplement that causes an optical density change. The TB, which is a diazo blue dye generally used to selectively colour dead tissues or cells, is a substrate in the present experiment. The concentration of the TB dissolving in water (TB solution) is 0.008% v/v. The GOQD may be preferably selected from nitrogen-doped graphene oxide quantum dot. The GOQD may be dissolved in the TB solution in a concentration range from about 1×10⁻¹⁵ milligram per milliliter (mg/mL) to about 500 milligram per milliliter (mg/mL); the GOQD concentration in the TB solution may be preferably used in a range from 1×10⁻¹⁰ milligram per milliliter (mg/mL) to about 50 mg/mL. The wavelength of the light supplement is used in a range from 200 nanometer to 1600 nanometer or any combination thereof; the wavelength of the light supplement is preferably used in a range from 400 nanometer to 1000 nanometer or any combination thereof. The illumination intensity of the light supplement may be irradiated in a range from about 1×10⁻⁶ microWatt per centimeter square (μWatt/cm²) to about 100 Watt per centimeter square (Watt/cm²); the illumination intensity of the light supplement may be preferably irradiated in a range from about 10 μWatt/cm² to about 5 Watt/cm². The illumination time of the light supplement is administered in a range from 1 femtosecond to 40 months; the illumination time of the light supplement is preferably administered in a range from about 1 micro-seconds to about 30 days. The optical density of the TB solution without the GOQD (marked TB in FIG. 1) under the light supplement is define as 0.7 relative unit (RU). The term “relative unit” is a unit of measurement used in analysis which employs optical density detection. The optical density of the TB solution with the GOQD (marked GOQD in FIG. 1) under the light supplement decreases to around 0.3 RU. The optical density of the TB solution with the TEOA under the light supplement (marked TEOA in FIG. 1) changes to around 0.7 RU. The optical density of the TB solution with the GOQD and the TEOA under the light supplement (marked GOQD+TEOA in FIG. 1) changes to around 0.15 RU. According to the above result, the GOQD can oxidize or reduce the TB to cause the optical density change of the TB solution. Furthermore, the chemical reactions can be enhanced in the presence of the TEOA.

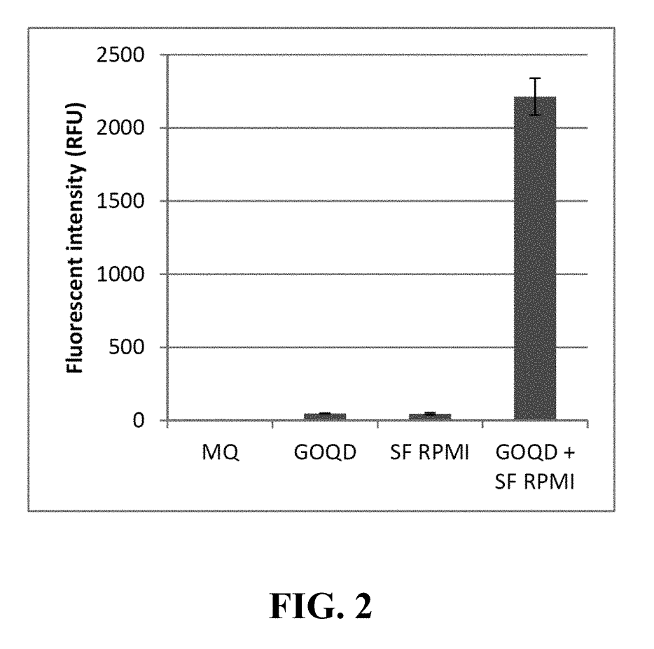

FIG. 2 illustrates fluorescent intensity changes of chemical reactions of Amplex® Red reagent dissolving in Milli-Q ultrapure water (MQ) without or with the GOQD at the concentration of 0.008 mg/mL, without or with an enhancer serum free RPMI (SF RPMI) at the concentration of 1×, or with a combination of the GOQD and the SF RPMI, under a light supplement (55 mWatt/cm², 10 min). The chemical reaction with the MQ is carried out to produce hydrogen peroxide (H₂O₂) in the presence of the GOQD under the light supplement. The Amplex Red reagent, a highly sensitive and stable probe for hydrogen peroxide (H₂O₂), is one of the best fluorogenic substrate for peroxidase. The H₂O₂ amount is analyzed using the Amplex® Red reagent which reacts with H₂O₂ in a 1:1 stoichiometry to produce highly fluorescent resorufin. The resorufin has maximum excitation and maximum emission approximately at 571 nanometer and 585 namometer, respectively. Thus, the output of the chemical reactions in the present experiments can be evaluated through the fluorescence output. The concentration of the Amplex® Red reagent in Milli-Q ultrapure water (MQ solution) is 100 micromoles (μM). The GOQD may be preferably selected from nitrogen-doped graphene oxide quantum dot. The GOQD is dissolved in the MQ solution in a concentration range from about 1×10⁻¹⁵ milligram per milliliter (mg/mL) to about 500 milligram per milliliter (mg/mL); the GOQD a concentration in the MQ solution may be preferably used in a range from 1×10⁻¹⁰ milligram per milliliter (mg/mL) to about 50 mg/mL. The wavelength of the light supplement is used in a range from 200 nanometer to 1600 nanometer, or any combination thereof. The illumination intensity of the light supplement may be used in a range from about 1×10⁻⁶ μWatt/cm² to about 100 Watt/cm²; the illumination intensity of the light supplement may be preferably used in a range from about 10 μWatt/cm² to about 5 Watt/cm². The illumination time of the light supplement is administered in a range from 1 femtosecond to 7 days; the illumination time of the light supplement is preferably administered in a range from about 10 micro-seconds to about 30 hours. The fluorescent intensity of the chemical reaction of the MQ solution without the GOQD (marked MQ in FIG. 2) under the light supplement is around 0 relative fluorescence unit (RFU). The term “relative fluorescence unit” is a unit of measurement used in analysis which employs fluorescence detection. The fluorescent intensity of the chemical reaction of the MQ solution with the GOQD (marked GOQD in FIG. 2) under the light supplement is around 50 RFU. The fluorescent intensity of the chemical reaction of the MQ solution with the SF RPMI (marked SF RPMI in FIG. 2) under the light supplement is around 50 RFU. The fluorescent intensity of the chemical reaction of the MQ solution with the GOQD and the SF RPMI (marked GOQD+SF RPMI in FIG. 2) under the light supplement is around 2,300 RFU. According to the experimental results, the chemical reaction of the MQ with the GOQD is carried out to produce the H₂O₂ under the light supplement that causes fluorescent intensity changes. Furthermore, the chemical reactions can be enhanced in the presence of the SF RPMI.

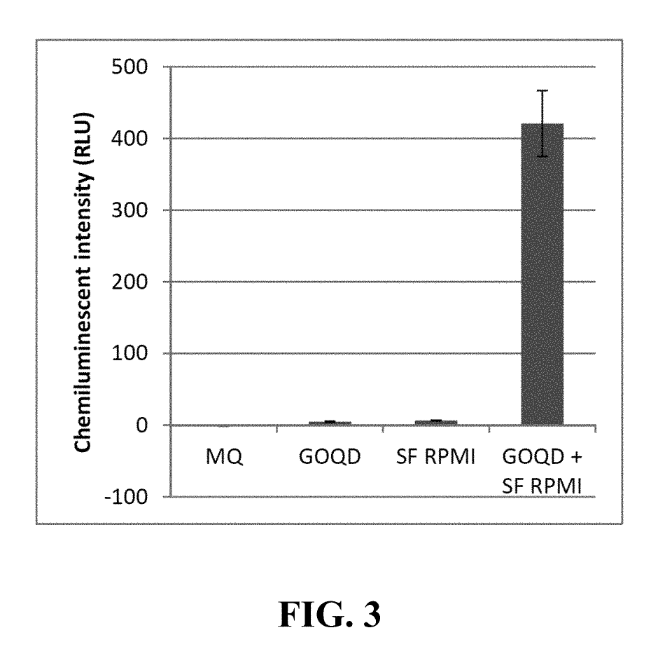

FIG. 3 shows chemiluminescent intensity changes of chemical reactions of a Luciferin Precursor reagent dissolving in Milli-Q ultrapure water (MQ) without or with graphene oxide quantum dots (GOQD) at the concentration of 0.008 mg/mL, without or with an enhancer serum free RPMI (SF RPMI) at the concentration of 1×, or with a combination of the GOQD and the SF RPMI, under a light supplement (55 mWatt/cm², 10 min). The chemical reaction with the MQ is carried out to produce hydrogen peroxide (H₂O₂) in the presence of the GOQD under the light supplement. The H₂O₂ amount is analyzed using the Luciferin Precursor reagent. The Luciferin Precursor reagent is employed as the H₂O₂ substrate that reacts directly with the H₂O₂ to generate a luciferin precursor. Upon addition of ROS-Glo™ Detection Reagent containing Ultra-Glo™ Recombinant Luciferase and d-Cysteine, the luciferin precursor is converted to a luciferin by the d-Cysteine, and the luciferin reacts with Ultra-Glo™ Recombinant Luciferase to generate a luminescent signal. The luminescent signal is proportional to the H₂O₂ concentration. Thus, the output of the chemical reactions in the present experiments can be evaluated through the luminescent signal output. The concentration of the Luciferin Precursor reagent in Milli-Q ultrapure water (MQ solution) is 1×. The 1× ROS-Glo™ Detection Reagent is added to the MQ solution. The GOQD may be preferably selected from nitrogen-doped graphene oxide quantum dot. The GOQD is dissolved in the MQ solution in a concentration range from about 1×10⁻¹⁵ milligram per milliliter (mg/mL) to about 500 mg/mL; a concentration may be preferably used in a range from 1×10⁻¹⁰ milligram per milliliter (mg/mL) to about 50 mg/mL. The wavelength of the light supplement is used in a range from 200 nanometer to 1600 nanometer or any combination thereof. The illumination intensity of the light supplement may be used in a range from about 1×10⁻⁶ μWatt/cm² to about 100 Watt/cm²; the illumination intensity of the light supplement may be preferably used in a range from about 10 μWatt/cm² to about 5 Watt/cm². The illumination time of the light supplement is administered in a range from 1 femtosecond to 7 days; the illumination time of the light supplement is preferably administered in a range from about 10 micro-seconds to about 30 hours. The chemiluminescent intensity of the chemical reaction of the MQ solution without the GOQD (marked MQ in FIG. 3) under the light supplement is about 0 relative luminescence unit (RLU). The term “relative luminescence unit” is a unit of measurement used in analysis which employs luminescence detection. The chemiluminescent intensity of the chemical reaction of the MQ solution with the GOQD (marked GOQD in FIG. 3) under the light supplement is around 5 RLU. The chemiluminescent intensity of the chemical reaction of the MQ solution with the SF RPMI (marked SF RPMI in FIG. 3) under the light supplement is around 5 RLU. The chemiluminescent intensity of the chemical reaction of the MQ with the GOQD and the SF RPMI (marked GOQD+SF RPMI in FIG. 3) under the light supplement is around 420 RLU. According to the experimental results, the chemical reaction of the MQ with the GOQD is carried out to produce the H₂O₂ under the light supplement that causes chemiluminescent intensity changes. Furthermore, the chemical reactions can be enhanced in the presence of the SF RPMI.

The fluorescent intensity changes in the chemical reactions presented in FIG. 2 or the chemiluminescent intensity changes in the chemical reactions presented in FIG. 3 may be carried out by the GOQD in an indirect manner through a specific mediator-H₂O₂. The fluorescent intensity changes or the chemiluminescent intensity changes in the chemical reactions can be enhanced by the presence of an enhancer, for example the SF RPMI.

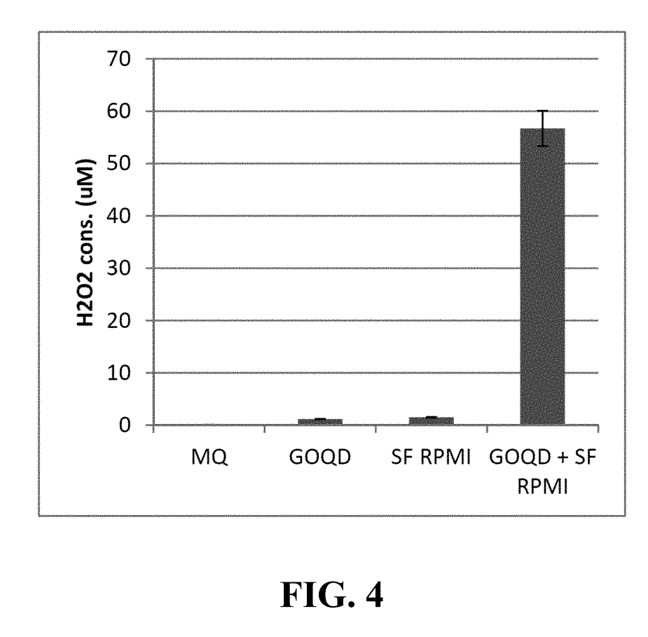

FIG. 4 illustrates concentration (conc., in micro molar (μM)) changes of the specific mediator H₂O₂ in chemical reactions presented in FIG. 2. The chemical reaction with the Milli-Q ultrapure water is carried out to produce H₂O₂ with or without GOQD under a light supplement. The H₂O₂ concentration in the chemical reactions of the Milli-Q ultrapure water without the GOQD (marked MQ in FIG. 4) under the light supplement is produced about 0 μM. The H₂O₂ concentration in the chemical reactions of the Milli-Q ultrapure water with the GOQD (marked GOQD in FIG. 4) under the light supplement is around 1 μM. The H₂O₂ concentration in the chemical reactions of the Milli-Q ultrapure water with the GOQD and the SF RPMI (marked GOQD+SF RPMI in FIG. 4) under the light supplement is around 55 μM. The H₂O₂ concentration produced in the GOQD+SF RPMI condition is 50 folds than in the GOQD condition. Thus, the enhancer, for example the SF RPMI, can be augment the chemical reactions to enhance the fluorescent intensity changes or the chemiluminescent intensity changes.

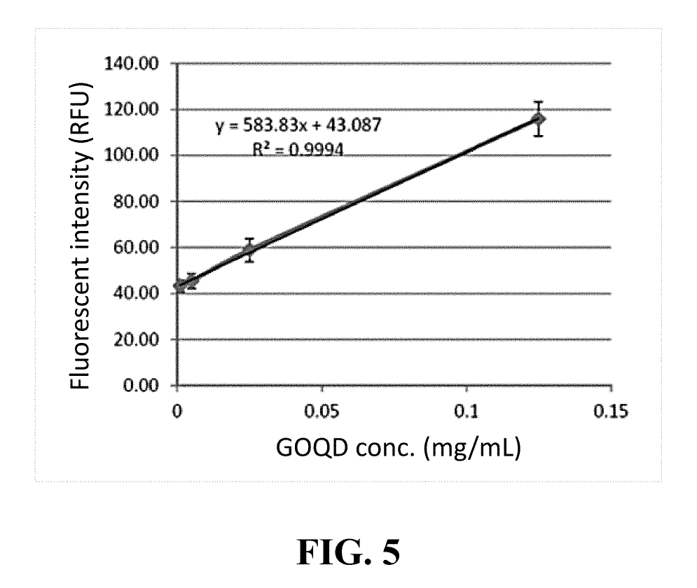

FIG. 5 shows illustrates a linear statistics result of chemical reactions which is correlated well with the concentrations (conc., in microgram per milliliter (mg/mL)) of the GOQD in a reaction system. The changes of optical density in FIG. 1, the fluorescence output in FIG. 2 or the luminescent signal output in FIG. 3 can be converted to the linear statistics formula shown in FIG. 5. In other words, the optical density, the fluorescence output or the luminescent signal output can be predicted with the different GOQD concentration added in the reaction system to use to an application comprising medical application and disintegrator application.

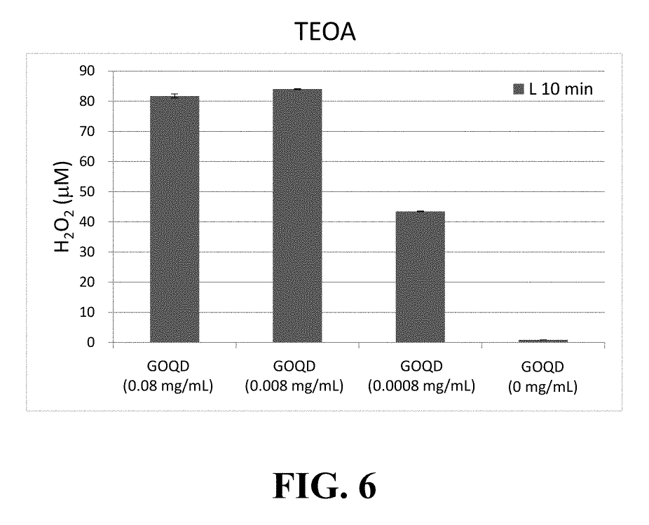

FIG. 6 illustrates hydrogen peroxide (H₂O₂) concentration (μM) changes of reaction systems under different doses of graphene oxide quantum dots (GOQD) ranging from 0.08 mg/mL to 0.0008 mg/mL, with an enhancer (triethanolamine, TEOA) at the concentration of 0.5% v/v, under a light supplement (55 mWatt/cm², 10 min). With the same rationale as FIG. 4, fluorescent intensity changes in the chemical reactions can be calculated and translated to the concentration changes of the H₂O₂. The H₂O₂ concentration in the chemical reactions of the Milli-Q ultrapure water with the GOQD and the TEOA (marked GOQD (0.08 mg/mL), GOQD (0.008 mg/mL), GOQD (0.0008 mg/mL) and GOQD (0 mg/mL) in FIG. 6) under the light supplement is around 80 μM, 80 μM, 40 μM, and 1 μM, respectively. The H₂O₂ concentration produced in the presence of the 0.5% v/v TEOA at the same GOQD concentration (0.008 mg/mL) is 80 μM which is 80 folds higher than that in the absence of the enhancer condition in FIG. 4 marked GOQD (around 1 μM H₂O₂). The concentration changes of the H₂O₂ in the chemical reactions can be enhanced by the presence of the TEOA in a various GOQD concentrations.

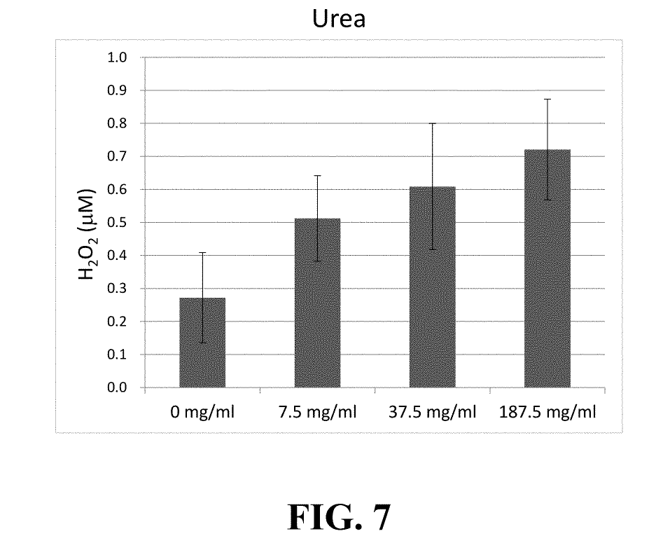

FIG. 7 illustrates hydrogen peroxide (H₂O₂) concentration (μM) changes of reaction systems under graphene oxide quantum dots (GOQD) at the concentration of 0.0008 mg/mL, with different doses of an enhancer (Urea) at the concentration of 7.5 to 187.5 mg/mL, under a light supplement (55 mWatt/cm², 10 min). With the same rationale as FIG. 4, fluorescent intensity changes in the chemical reactions can be calculated and translated to the concentration changes of the H₂O₂. The H₂O₂ concentration in the chemical reactions of the Milli-Q ultrapure water with the GOQD and the Urea (marked (0 mg/mL), (7.5 mg/mL), (37.5 mg/mL) and (187.5 mg/mL) in FIG. 7) under the light supplement is around 0.2 μM, 0.5 μM, 0.6 μM, and 0.7 μM, respectively. The H₂O₂ concentration produced in the presence of 187.5 mg/mL Urea at the same concentration of GOQD (0.0008 mg/mL) is 3.5 folds higher than that in the absence of the enhancer. The concentration changes of the H₂O₂ in the chemical reactions can be enhanced by the presence of the Urea.

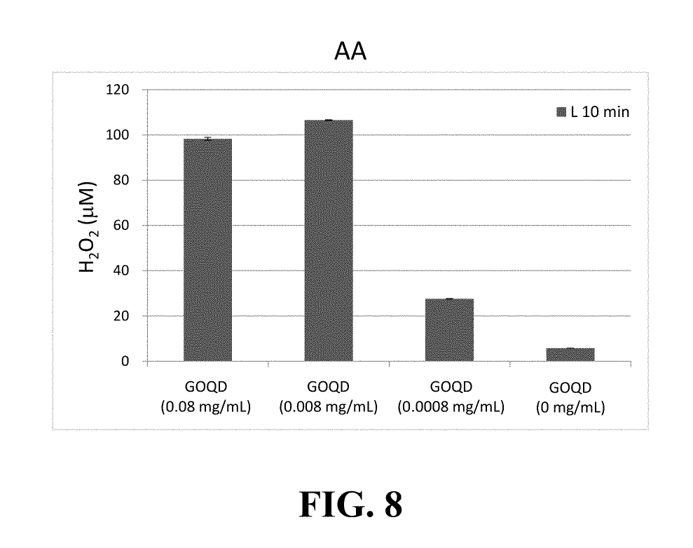

FIG. 8 illustrates hydrogen peroxide (H₂O₂) concentration (μM) changes of reaction systems under different doses of graphene oxide quantum dots (GOQD) ranging from 0.08 mg/mL to 0.0008 mg/mL, with an enhancer (ascorbic acid, AA) at the concentration of 2 mM, under a light supplement (55 mWatt/cm², 10 min). With the same rationale as FIG. 4, fluorescent intensity changes in the chemical reactions can be calculated and translated to the concentration changes of the H₂O₂. The H₂O₂ concentration in the chemical reactions of the Milli-Q ultrapure water with the GOQD and the AA (marked GOQD (0.08 mg/mL), GOQD (0.008 mg/mL), GOQD (0.0008 mg/mL) and GOQD (0 mg/mL) in FIG. 8) under the light supplement is around 100 μM, 110 μM, 30 μM, and 10 μM, respectively. The H₂O₂ concentration produced in the presence of 2 mM AA at the same GOQD concentration (0.008 mg/mL) is 110 μM which is 110 folds higher than that in the absence of the specific enhancer condition in FIG. 4 marked GOQD (around 1 μM H₂O₂). The concentration changes of the H₂O₂ in the chemical reactions can be enhanced by the presence of the AA.

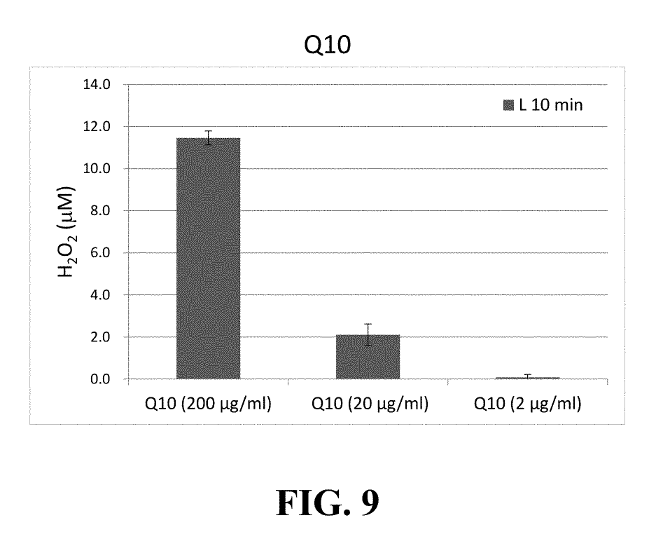

FIG. 9 illustrates hydrogen peroxide (H₂O₂) concentration (μM) changes of reaction systems under graphene oxide quantum dots (GOQD) at the concentration of 0.0008 mg/mL, with different doses of a specific enhancer (co-enzyme Q10, Q10), at the concentration of 2 to 200 μg/mL, under a light supplement (55 mW/cm², 10 min). With the same rationale as FIG. 4, fluorescent intensity changes in the chemical reactions can be calculated and translated to the concentration changes of the H₂O₂. The H₂O₂ concentration in the chemical reactions of the Milli-Q ultrapure water with the GOQD and the Q10 (marked (200 μg/mL), (20 μg/mL), and (2 μg/mL) in FIG. 9) under the light supplement is around 12 μM, 2 μM, and 0.2 μM, respectively. The H₂O₂ concentration produced in the presence of 200 μg/mL Q10 at the same GOQD concentration (0.0008 mg/mL) is 12 μM, which is 60 folds higher than that in the absence of the specific enhancer condition in FIG. 7 marked 0 mg/mL (around 0.2 μM H₂O₂). The concentration changes of the H₂O₂ in the chemical reactions can be enhanced by the presence of the Q10.

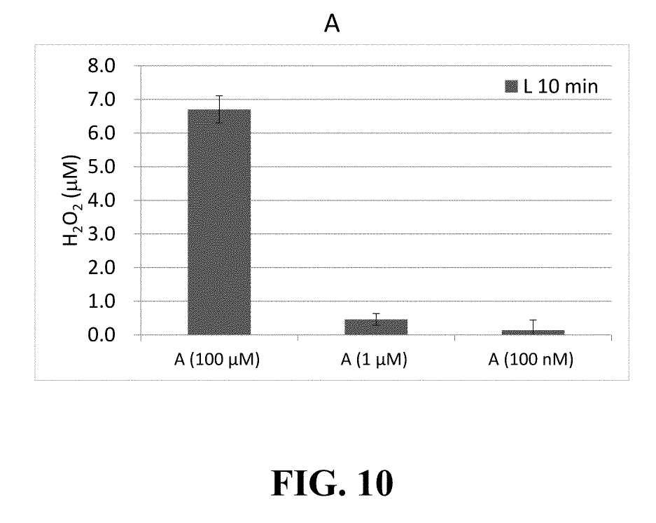

FIG. 10 illustrates hydrogen peroxide (H₂O₂) concentration (μM) changes of reaction systems under graphene oxide quantum dots (GOQD) on the concentration of 0.0008 mg/mL, with different doses of an enhancer (astaxanthin, A), on the concentration of 0.1 μM to 100 μM, under a light supplement (55 mWatt/cm², 10 min). With the same rationale as FIG. 4, fluorescent intensity changes in the chemical reactions can be calculated and translated to the concentration changes of the H₂O₂. The H₂O₂ concentration in the chemical reactions of the Milli-Q ultrapure water with the GOQD and the A (marked (100 μM), (1 μM), and (100 nM) in FIG. 10) under the light supplement is around 7 μM, 0.5 μM, and 0.2 μM, respectively. The H₂O₂ concentration produced in the presence of 100 μM A at the same GOQD concentration (0.0008 mg/mL) is 7 μM, which is 35 folds higher than that in the absence of the specific enhancer condition in FIG. 7 marked 0 mg/mL (around 0.2 μM H₂O₂). The concentration changes of the H₂O₂ in the chemical reactions can be enhanced by the presence of the A.

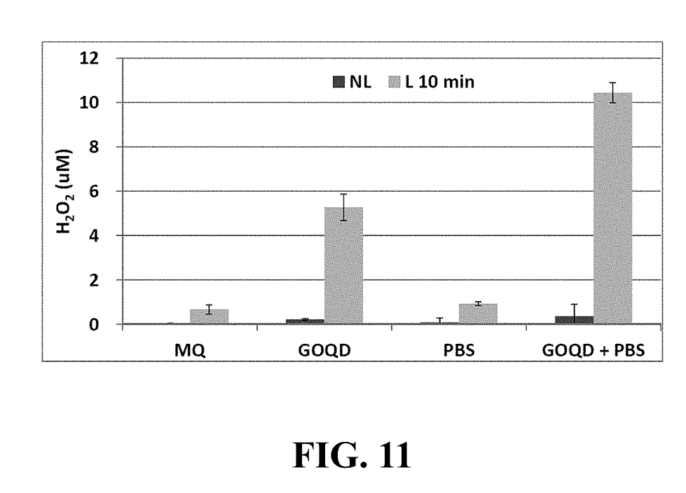

FIG. 11 illustrates concentration hydrogen peroxide (H₂O₂) (μM) changes of reaction systems without or with graphene oxide quantum dots (GOQD) at the concentration of 0.08 mg/mL, without or with an enhancer (phosphate-buffered saline, PBS) at the concentration of 1×, or with a combination of the GOQD and the PBS, without or with a light supplement (55 mWatt/cm², 10 min). With the same rationale as FIG. 4, fluorescent intensity changes in the chemical reactions can be calculated and translated to the concentration changes of the H₂O₂. The H₂O₂ concentration in the chemical reactions of the Milli-Q ultrapure water without the GOQD (marked MQ), with the GOQD (marked GOQD), with the PBS (marked PBS), or the combination of the GOQD and the PBS (marked GOQD+PBS) under the light supplement is around 0.5 μM, 5 μM, 1 μM, and 10 μM, respectively. Without the light supplement almost no H₂O₂ is detected. At the same concentration of GOQD (0.08 mg/mL), the H₂O₂ concentration produced in the presence of the PBS is 2 folds higher than that in the absence of the enhancer. The concentration changes of the H₂O₂ in the chemical reactions can be enhanced by the presence of the PBS.

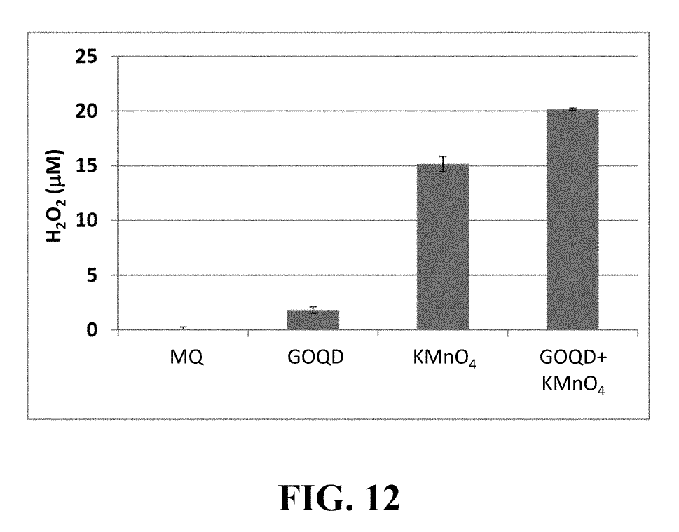

FIG. 12 illustrates hydrogen peroxide (H₂O₂) concentration (μM) changes of reaction systems without or with graphene oxide quantum dots (GOQD) at the concentration of 0.008 mg/mL, without or with an enhancer (KMnO₄) at the concentrate of 0.1 mM, or with a combination of the GOQD and the KMnO₄, under a light supplement (55 mWatt/cm², 10 min). With the same rationale as FIG. 4, fluorescent intensity changes in the chemical reactions can be calculated and translated to the concentration changes of the H₂O₂. The H₂O₂ concentration in the chemical reactions of the Milli-Q ultrapure water without the GOQD (marked MQ), with the GOQD (marked GOQD), with the KMnO₄ (marked KMnO₄), or with the combination of the GOQD and the KMnO₄ (marked GOQD+KMnO₄) under the light supplement is around 0 μM, 1 μM, 15 μM, and 20 μM, respectively. Without the light supplement almost no H₂O₂ is detected. At the same concentration of GOQD (0.008 mg/mL), the H₂O₂ concentration produced in the presence of the KMnO₄ is 20 folds higher than that in the absence of the enhancer. The concentration changes of the H₂O₂ in the chemical reactions can be enhanced by the presence of the KMnO4.

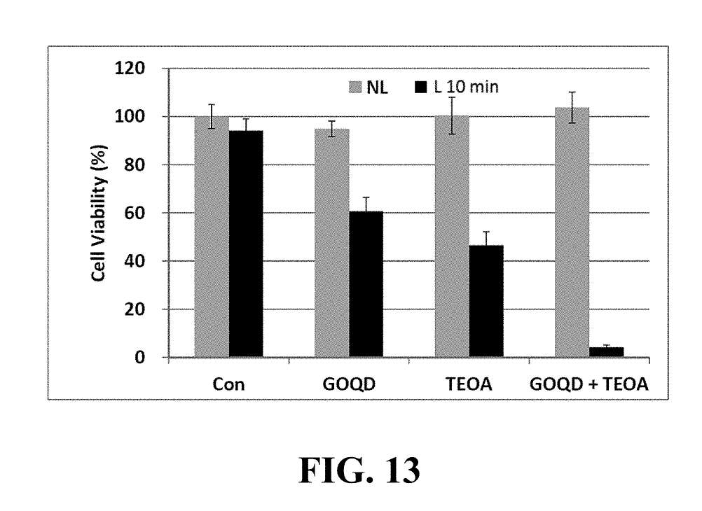

FIG. 13 illustrates substrate activities changes treating without or with graphene oxide quantum dots (GOQD) at the concentration of 0.008 mg/mL, without or with an enhancer (triethanolamine, TEOA) at the concentration of 0.05% v/v, or with a combination of the GOQD and the TEOA, and without or with a light supplement (55 mWatt/cm², 10 min). The substrate activities here are refer to the cell viability (%) of lung cancer cells (PC-9) and are analyzed using MTT assay for assessing cell metabolic activity that NAD(P)H-dependent cellular oxidoreductase enzymes may, under defined conditions, reflect the number of viable cells present. These enzymes are capable of reducing the tetrazolium dye MTT (3-(4,5-dimethylthiazol-2-yl)-2,5-diphenyltetrazolium bromide) to its insoluble formazan, which has a purple color. A solubilization solution (dimethyl sulfoxide, DMSO) is added to dissolve the insoluble purple formazan product into a colored solution. The absorbance of this colored solution can be quantified by measuring at a certain wavelength (490 nm) by a spectrophotometer and represent the viability of the cells. The final MTT concentration in the analysis is 0.5 mg/mL and the DMSO is added at the concentration of 10% v/v. The GOQD may be preferably selected from nitrogen-doped graphene oxide quantum dot. The GOQD is dissolved in the culture medium in a concentration range from about 1×10⁻¹⁵ milligram per milliliter (mg/mL) to about 500 mg/mL; a concentration may be preferably used in a range from 1×10⁻¹⁰ milligram per milliliter (mg/mL) to about 50 mg/mL. The wavelength of the light supplement is used in a range from 200 nanometer to 1600 nanometer or any combination thereof. The illumination intensity of the light supplement may be used in a range from about 1×10⁻⁶ μWatt/cm² to about 100 Watt/cm²; the illumination intensity of the light supplement may be preferably used in a range from about 10 μWatt/cm² to about 5 Watt/cm². The illumination time of the light supplement is administered in a range from 1 femtosecond to 7 day; the illumination time of the light supplement is preferably administered in a range from about 10 micro-seconds to about 20 hours. The changes of substrate activities (cell viability (%)) of the reaction system, without the GOQD or the TEOA (marked Con in FIG. 13) and without the light supplement is defined as 100%. Via the normalization, the cell viability (%) of the reaction system with the GOQD (marked GOQD in FIG. 13) and under the light supplement (marked L10 min) is around 60%. The change of the cell viability (%) of the reaction system with the TEOA (marked TEOA in FIG. 13) and under the light supplement is around 50%. The change of the cell viability (%) of the reaction system with the GOQD and the TEOA (marked GOQD+TEOA in FIG. 13) and under the light supplement is around 10%. The substrate activity changes in the reaction system can be enhanced by the presence of the TEOA. Almost no change of the substrate activity changes are found in the groups without light supplement (marked NL).

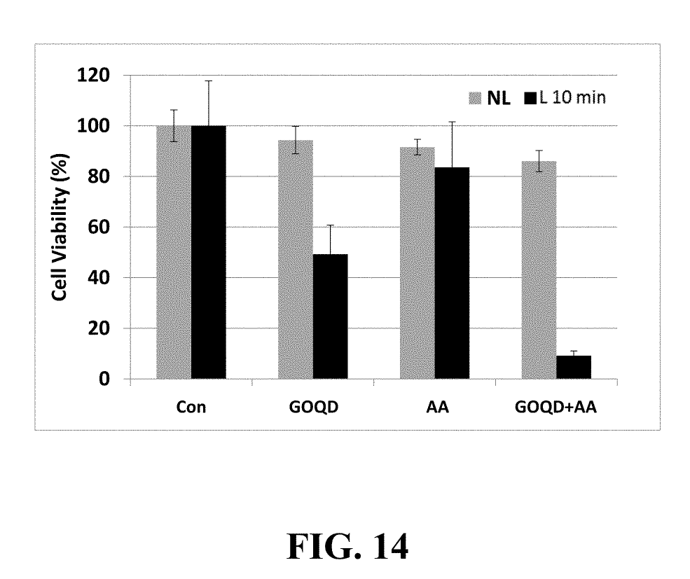

FIG. 14 illustrates change of substrate activities of reaction systems in which the substrates are lung cancer cells (PC-9) without or with graphene oxide quantum dots (GOQD) at the concentration of 0.008 mg/mL, without or with an enhancer (ascorbic acid, AA) at the concentration of 0.05 mM, or with a combination of the GOQD and the AA, and without or with a light supplement (55 mWatt/cm², 10 min). The substrate activities here are refer to the cell viability (%) and are analyzed using MTT assay. The final MTT concentration in the analysis is 0.5 mg/mL and the DMSO is added at the concentration of 10% v/v. The change of substrate activities (cell viability (%)) of the reaction system, without the GOQD or the AA (marked Con in FIG. 14) and without the light supplement (marked NL) is defined as 100%. Via the normalization, the cell viability (%) of the reaction system with the GOQD (marked GOQD in FIG. 13) under the light supplement is around 50%. The change of the cell viability (%) of the reaction system with the AA (marked AA in FIG. 13) and under the light supplement (marked L10 min) is around 80%. The change of the cell viability (%) of the reaction system with the GOQD and the TEOA (marked GOQD+TEOA in FIG. 13) and under the light supplement is around 10%. The substrate activity changes in reaction system can be enhanced by the presence of the AA. Only limited change of the substrate activity changes are found in the groups without light supplement (marked NL).

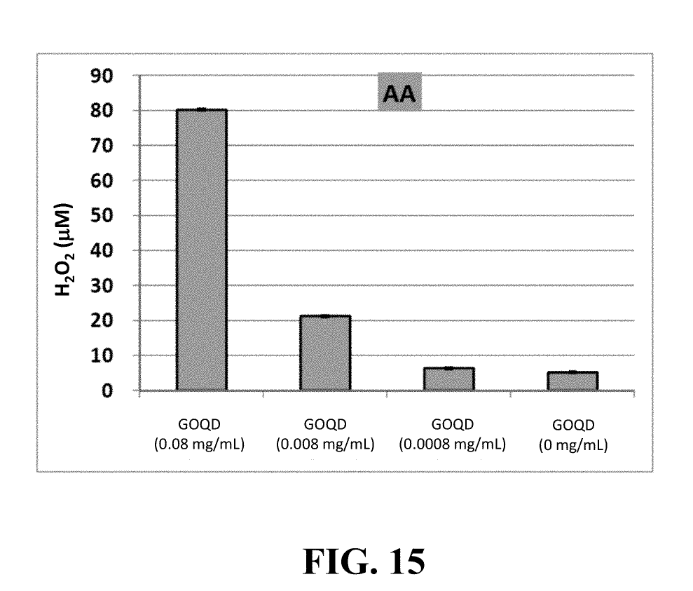

FIG. 15 illustrates hydrogen peroxide (H₂O₂) concentration (μM) changes of reaction systems under different doses of graphene oxide quantum dots (GOQD) ranging from 0.08 mg/mL to 0.0008 mg/mL, with an enhancer (ascorbic acid, AA) at the concentration of 2 mM, and without light supplement. With the same rationale as FIG. 4, fluorescent intensity changes in the chemical reactions can be calculated and translated to the concentration changes of the H₂O₂. The H₂O₂ concentration in the chemical reactions of the Milli-Q ultrapure water with the GOQD and the AA (marked GOQD (0.08 mg/mL), GOQD (0.008 mg/mL), GOQD (0.0008 mg/mL) and GOQD (0 mg/mL) in FIG. 15) without light supplement is around 80 μM, 20 μM, 10 μM, and 5 μM, respectively. The H₂O₂ concentration produced in the presence of 2 mM AA at the same GOQD concentration (0.08 mg/mL) is 80 μM which is 16 folds higher than that in the absence of the specific enhancer condition in FIG. 11 marked GOQD (around 5 μM H₂O₂). The concentration changes of the H₂O₂ in the chemical reactions can be enhanced by the presence of the AA not only in the presence of predetermined energy but also in the absence of predetermined energy.

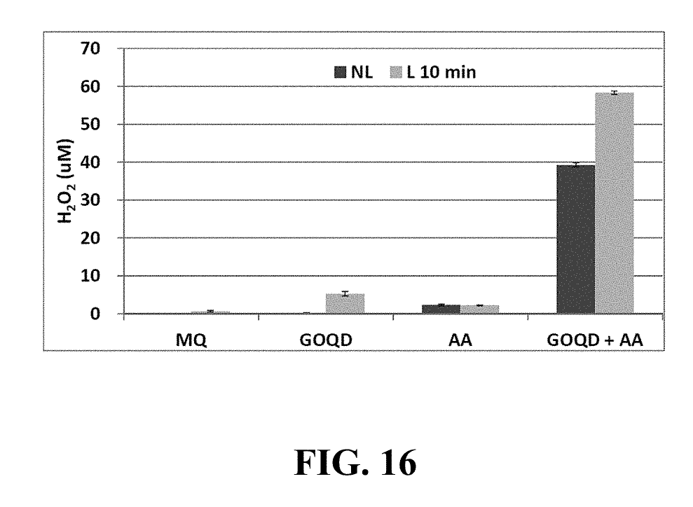

FIG. 16 illustrates hydrogen peroxide (H₂O₂) concentration (μM) changes of reaction systems without or with graphene oxide quantum dots (GOQD) at the concentration of 0.08 mg/mL, without or with an enhancer (ascorbic acid, AA) at the concentration of 0.2 mM, or with a combination of the GOQD and the AA, and without or with a light supplement (55 mWatt/cm², 10 min). With the same rationale as FIG. 4, fluorescent intensity changes in the chemical reactions can be calculated and translated to the concentration changes of the H₂O₂. The H₂O₂ concentration in the chemical reactions of the Milli-Q ultrapure water without the GOQD (MQ) or with the GOQD (GOQD), with the AA (AA), or with the combination of the GOQD and the AA (GOQD+AA) under the light supplement is around 15 μM, 5 μM, 2 μM, and 60 μM, respectively. Without the light supplement, without the GOQD (MQ) or with the GOQD (GOQD), with the AA (AA), or with the combination of the GOQD and the AA (GOQD+AA) under the light supplement is around 0 μM, 0 μM, 2 μM, and 40 μM, respectively. In the present of the GOQD (0.08 mg/mL), the H₂O₂ concentration produced under the light supplement is 1.5 folds higher than that in the absence of under the light supplement. The concentration changes of the H₂O₂ in the chemical reactions can be enhanced by the presence of the AA in the absence of light supplement which can be further augmented by the light supplement.

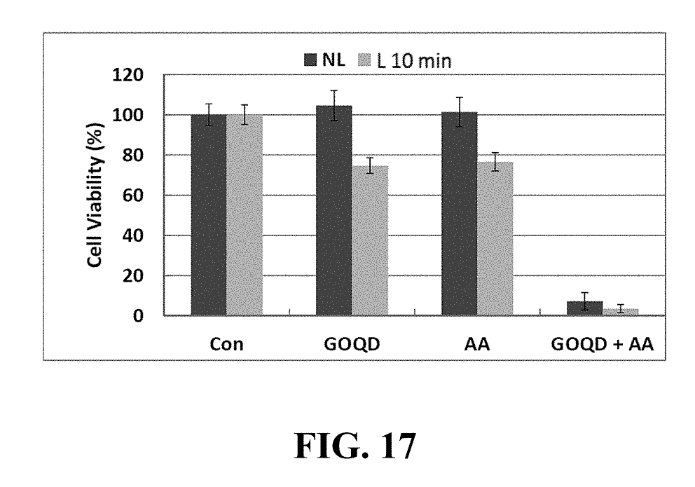

FIG. 17 illustrates change of substrate activities of reaction systems in which the substrates are lung cancer cells (PC-9) without or with graphene oxide quantum dots (GOQD) at the concentration of 0.08 mg/mL, without or with an enhancer (ascorbic acid, AA) at the concentration of 0.2 mM, or with a combination of the GOQD and the AA, and without or with a light supplement (55 mWatt/cm² for 10 min). The substrate activities here are refer to the cell viability and are analyzed using MTT assay. The final MTT concentration in the analysis is 0.5 mg/mL and the DMSO is added at the concentration of 10% v/v. The change of substrate activities (cell viability (%)) of the reaction system, without the GOQD or the AA (marked Con in FIG. 17) and without the light supplement (marked NL) is defined as 100%. Via the normalization, the cell viability (%) of the reaction system with the GOQD (marked GOQD in FIG. 17) under the light supplement is around 70%. The change of cell viability (%) of the reaction system with the AA (marked AA in FIG. 17) under the light supplement (marked L10 min) is around 80%. The change of cell viability (%) of the system with the GOQD and the AA (marked GOQD+AA in FIG. 17) under the light supplement is around 5%. The substrate activity changes in the reaction system can be enhanced by the presence of the AA. Almost no change of the substrate activity changes are found in the groups without light supplement (marked NL) except the system with the GOQD and the enhancer-AA which indicated that the substrate activity changes in the reaction system can be enhanced by the presence of the enhancer-AA in the absence of light supplement in the high concentration condition (comparing with the lower on in FIG. 14).

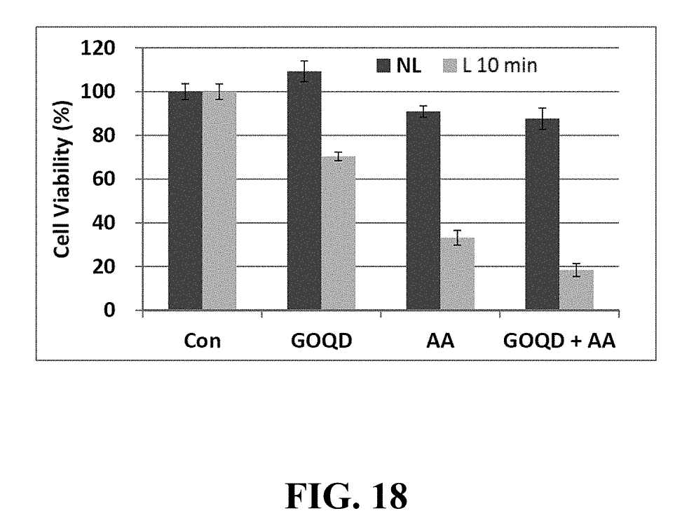

FIG. 18 illustrates change of substrate activities of reaction systems in which the substrates are colon cancer cells (HCT-116) without or with graphene oxide quantum dots (GOQD) at the concentration of 0.08 mg/mL, without or with an enhancer (ascorbic acid, AA) at the concentration of 0.2 mM, or with a combination of the GOQD and the AA, and without or with a light supplement (55 mWatt/cm², 10 min). The substrate activities here are refer to the cell viability and are analyzed using MTT assay. The final MTT concentration in the analysis is 0.5 mg/mL and the DMSO is added at the concentration of 10% v/v. The change of substrate activities (cell viability (%)) of the reaction system, without the GOQD or the AA (marked Con in FIG. 18) and without the light supplement (marked NL) is defined as 100%. Via the normalization, the cell viability (%) of the reaction with the GOQD (marked GOQD in FIG. 18) under the light supplement is around 70%. The change of cell viability (%) of the reaction system with the AA (marked AA in FIG. 18) under the light supplement (marked L10 min) is around 40%. The change of cell viability (%) of the system with the GOQD and the AA (marked GOQD+AA in FIG. 18) under the light supplement is around 10%. The substrate activity changes in the reaction system can be enhanced by the presence of the AA. Almost no change of the substrate activity changes are found in the groups without light supplement (marked NL) except the system with the GOQD and the enhancer-AA. These results indicated that the change of the substrate activities of the reaction system which the substrates could not only be lung cancer cells but also colon cancer cells.

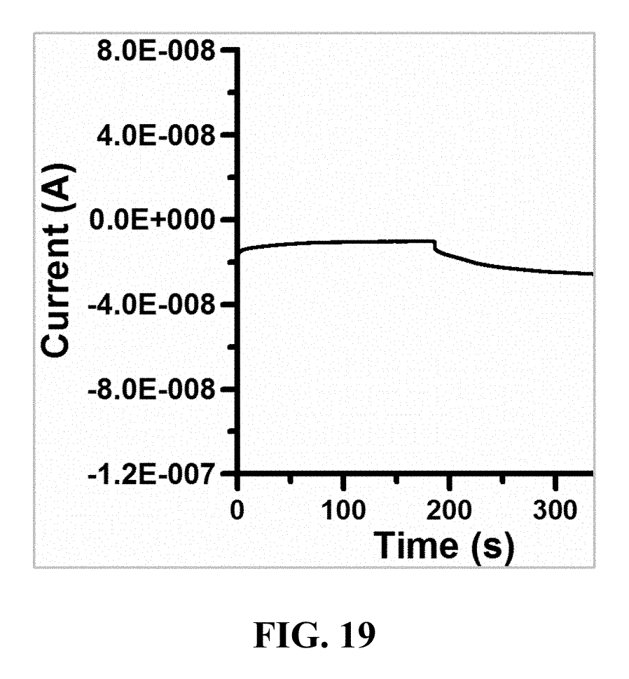

FIG. 19 illustrates current intensity changes of a reaction system with graphene oxide quantum dots (GOQD) on an electron conductive electrode, immersed in an enhancer (triethanolamine, TEOA) at the concentration of 0.05% v/v, and without or with an applied voltage (0.1 Volt) and a light supplement (35 mWatt/cm², 3 min). The current intensity changes are analyzed using potentiostat/galvanostat/cyclic voltammetry instruments on a gold coated slide covered with the GOQD. The GOQD may be preferably select from nitrogen-doped graphene oxide quantum dots. The wavelength of the light supplement is used in a range from 200 nanometer to 1600 nanometer or any combination thereof. The illumination intensity of the light supplement is used in a range from about 1×10⁻⁶ μWatt/cm² to about 100 Watt/cm²; the illumination intensity of the light supplement may be preferably irradiated in a range from about 10 μWatt/cm² to about 5 Watt/cm². The illumination time of the light supplement is administered in a range from 1 femtosecond to 1 day; the illumination time of the light supplement is preferably administered in a range from about 10 micro-seconds to about 20 hours. The change of current intensity of the reaction system is not found in the first 3 min without the light supplement. With the light supplement, a significant change of current intensity of reaction system is found. These results indicated the change in the reaction system can occur as a photoelectrochemical reaction and can be detected using potentiostat/galvanostatt/cyclic voltammetry instruments. Turning off the light reveal the change of current intensity from GOQD electrode. These results further imply an photoelectrochemical reaction induced by instantaneous light irradiation, and the variation of current intensity can be detected by using potentiostat/galvanostat/cyclic voltammetry instruments.

The embodiments shown and described above are only examples. Many details are often found in the art such as the other features of a graphene oxide quantum dots. Therefore, many such details are neither shown nor described. Even though numerous characteristics and advantages of the present technology have been set forth in the foregoing description, together with details of the structure and function of the present disclosure, the disclosure is illustrative only, and changes may be made in the detail, including in matters of type, size, concentration and arrangement of the parts within the principles of the present disclosure up to, and including the full extent established by the broad general meaning of the terms used in the claims. It will therefore be appreciated that the embodiments described above may be modified within the scope of the claims.

What is claimed is:

1. A reaction system for augmenting chemical reaction, photoelectrochemical reaction, photochemical reaction, electrochemical reaction or any combination thereof, comprising:
at least one additive, wherein the at least one additive is a kind of reaction enhancer; and
at least one reaction base-plane.

2. The reaction system of claim 1, further comprising at least one reaction substrate in the reaction system, the at least one reaction substrate comprises at least one inorganic compound, at least one organic compound, at least one bio-compound, at least one cell, at least one microbial, at least one parasite or any combination thereof.

3. The reaction system of claim 1, wherein at least one result of the chemical reaction, the photoelectrochemical reaction, the photochemical reaction, electrochemical reaction or any combination thereof comprises color change, luminescence intensity variation, fluorescence intensity variation, optical density variation, luminescence wavelength profile variation, fluorescence wavelength profile variation, optical wavelength profile variation, current intensity change, electron response, substrate amounts decreasing, substrate activities change, substrate compounds modification or substrate functions change or any combination thereof.

4. The reaction system of claim 1, wherein the chemical reaction, the photoelectrochemical reaction, the photochemical reaction, electrochemical reaction or any combination thereof may induce redox reaction, electrochromic, electrochemiluminescence, photochemiluminescence, or photoelectrochemiluminescence.

5. The reaction system of claim 1, wherein the chemical reaction, the photoelectrochemical reaction, the photochemical reaction or electrochemical reaction, or any combination thereof may be carried out in an indirect manner through a specific mediator including but not limited to H₂O₂.

6. The reaction system of claim 1, wherein the concentration of the at least one additive is used in the reaction system in a range from about 1×10⁻¹² volume per volume percentage (% v/v) to about 50 volume per volume percentage (% v/v) or a concentration range from about 1×10⁻¹⁵ molarity (M) to about 10 M.

7. The reaction system of claim 1, wherein the at least one additive comprises nutrients, vitamins, alkali salts, alkali buffers, organic compounds, inorganic compounds or transition metal ions having an empty d, f, or g orbital.

8. The reaction system of claim 1, wherein the at least one additive comprises serum free RPMI, serum free DMEM, serum free MEMα, serum free F12, serum free L15, serum free Hybri-Care, ascorbic acid, co-enzyme Q10, glutathione, astaxanthin, fetal bovine serum, phosphate-buffered saline, polysulfide, porphyrin, chlorophyll, histamine, methanol, ethanol, lactic acid, triethanolamine, silver nitrate, sodium iodate, ferric ion, ferrous, potassium permanganate, cobalt ion, nickel ion or any combination thereof.

9. The reaction system of claim 1, wherein the concentration of the at least one reaction base-plane is used in the reaction system in a range from about 1×10⁻¹⁵ milligram per milliliter (mg/mL) to 500 milligram per milliliter (mg/mL).

10. The reaction system of claim 1, wherein the at least one reaction base-plane comprises at least one non-metallic semiconductor quantum dot.

11. The reaction system of claim 10, wherein the at least one non-metallic semiconductor quantum dot comprises graphene quantum dot or graphene oxide quantum dot.

12. The reaction system of claim 10, wherein the at least one non-metallic semiconductor quantum dot further comprises at least one dopant.

13. The reaction system of claim 12, wherein the at least one dopant comprises at least one IIA group element, at least one IIIA group element, at least one IVA group element, at least one VA group element, at least one VIA group element, at least one transition element having an empty d orbital or any combination thereof.

14. The reaction system of claim 10, the at least one non-metallic semiconductor quantum dot further comprises at least one functional group, wherein the at least one functional group is located on the at least one non-metallic semiconductor quantum dot surface; wherein the at least one functional group is located in the at least one non-metallic semiconductor quantum dot; wherein the at least one functional group is located on and in the at least one non-metallic semiconductor quantum dot.

15. The reaction system of claim 14, wherein the at least one functional group comprises at least one hydrogen atom, at least one IIIA-element functional group, at least one IVA-element functional group, at least one VA-element functional group, at least one VIA-element functional group, VIIA-element functional group or any combination thereof.

16. A method, comprising:
providing at least one additive, wherein the at least one additive is a kind of reaction enhancer;
providing at least one reaction base-plane;
providing at least one reaction substrate; and
producing at least one chemical reaction result, at least one photoelectrochemical reaction result, at least one photochemical reaction result, at least one electrochemical reaction result or any combination thereof.

17. The method of claim 16, further comprising providing at least one predetermined energy before producing the at least one chemical reaction result, the at least one photoelectrochemical reaction result, the at least one photochemical reaction result, the at least one electrochemical reaction result or any combination thereof.

18. The reaction system of claim 16, wherein the at least one chemical reaction result, the at least one photoelectrochemical reaction result, the at least one photochemical reaction result, the at least one electrochemical reaction result or any combination thereof may comprise redox reaction, electrochromic, electrochemiluminescence, photochemiluminescence or photoelectrochemiluminescence.

19. The reaction system of claim 16, wherein the chemical reaction, the photoelectrochemical reaction, the photochemical reaction or electrochemical reaction, or any combination thereof may be carried out in an indirect manner through a specific mediator including but not limited to H₂O₂.

20. The reaction system of claim 16, wherein the concentration of the at least one additive is used in a range from about 1×10⁻¹² volume per volume percentage (% v/v) to about 50 volume per volume percentage (% v/v) or a concentration range from about 1×10⁻¹⁵ molarity (M) to about 10 M.

21. The method of claim 16, wherein the at least one additive comprises nutrients, vitamins, alkali salts, alkali buffers, organic compounds, inorganic compounds or transition metal ions having an empty d, f, or g orbital.

22. The reaction system of claim 16, wherein the at least one additive comprises serum free RPMI, serum free DMEM, serum free MEMα, serum free F12, serum free L15, serum free Hybri-Care, ascorbic acid, co-enzyme Q10, glutathione, astaxanthin, fetal bovine serum, phosphate-buffered saline, polysulfide, porphyrin, chlorophyll, histamine, methanol, ethanol, lactic acid, triethanolamine, silver nitrate, sodium iodate, ferric ion, ferrous, potassium permanganate, cobalt ion, nickel ion or any combination thereof.

23. The method of claim 16, wherein the concentration of the at least one reaction base-plane is used in a range from about 1×10⁻¹⁵ milligram per milliliter (mg/mL) to 500 milligram per milliliter (mg/mL).

24. The method of claim 16, wherein the at least one reaction base-plane comprises at least one non-metallic semiconductor quantum dot.

25. The method of claim 16, wherein the at least one reaction substrate comprises at least one inorganic compound, at least one organic compound, at least one bio-compound, at least one cell, at least one microbial, at least one parasite or any combination thereof.

26. The method of claim 24, wherein the at least one non-metallic semiconductor quantum dot comprises graphene quantum dot or graphene oxide quantum dot.

27. The method of claim 24, wherein the at least one non-metallic semiconductor quantum dot further comprises at least one dopant.

28. The method of claim 27, wherein the at least one dopant comprises at least one IIA group element, at least one IIIA group element, at least one IVA group element, at least one VA group element, at least one VIA group element, at least one transition element having an empty d orbital or any combination thereof.

29. The method of claim 24, the at least one non-metallic semiconductor quantum dot further comprises at least one functional group, wherein the at least one functional group is located on the at least one non-metallic semiconductor quantum dot surface; wherein the at least one functional group is located in the at least one non-metallic semiconductor quantum dot; wherein the at least one functional group is located on and in the at least one non-metallic semiconductor quantum dot.

30. The method of claim 29, wherein the at least one functional group comprises at least one hydrogen atom, at least one IIIA-element functional group, at least one IVA-element functional group, at least one VA-element functional group, at least one VIA-element functional group, at least one VIIA-element functional group or any combination thereof.

31. The method of claim 17, wherein the at least one predetermined energy comprises a radiation energy, a heat energy, an electrical energy, a magnetic energy or a mechanical energy or any combination thereof.

32. The method of claim 16, wherein the at least one chemical reaction result, the at least one photoelectrochemical reaction result, the at least one photochemical reaction result or the at least one electrochemical reaction result comprises color change, luminescence intensity variation, fluorescence intensity variation, optical density variation, luminescence wavelength profile variation, fluorescence wavelength profile variation, optical wavelength profile variation, current intensity change, electronic response, substrate amounts decreasing, substrate activities change, substrate compounds modification or substrate functions change or any combination thereof.

33. The method of claim 16, wherein the at least one photoelectrochemical reaction result comprises variation of photoluminescence reaction or variation of photovoltaic reaction.

34. The method of claim 16, wherein the at least one photochemical reaction result comprises variation of photocatalysis reaction.
